(19) United States
(12) Patent Application Publication
(10) Pub. No.: US 2008/0109010 A1
(43) Pub. Date: May 8, 2008

(54) Apparatus for cervical manipulation and methods of use

(75) Inventors: Gerald Feuer, Atlanta, GA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: FEMSUITE, LLC, San Francisco, CA (US)

(21) Appl. No.: 11/594,323

(22) Filed: Nov. 7, 2006

(51) Int. Cl. A61B 17/42

(52) U.S. Cl. 606/119

(57) ABSTRACT

In one embodiment, an apparatus for cervical manipulation includes an inner tubular member manufactured from a bendable, semi-rigid material having an inflatable balloon disposed at the distal end and a flexible wire coupled to the wall of a lumen within the inner tubular member, and also includes an outer tubular member within which the inner tubular member reciprocates and which carries at the distal end an applicator member shaped to engage the external os of a cervix.

### FIELD OF THE INVENTION

The present invention relates to apparatus and methods for manipulating the female cervix during a variety of gynecological procedures. More particularly, the present invention relates to apparatus and methods that provide opposing pressures on the female cervix by compressing a balloon against the internal os and by contemporaneously anchoring an applicator member to the external os.

### BACKGROUND OF THE INVENTION

The uterine cervix is situated in the lower region of the uterus and not only connects the uterine cavity to the vagina, but also maintains the uterus in proper position within the pelvis due to a complex ligamentary structure named retinaculum uteri. The cervix of a female adult is typically four centimeters long, and in contrast to the uterine corpus, most of the cervix structure is formed by dense collagenous tissue containing a small amount of smooth muscle fibers.

Gynecological and obstetrical procedures may require manipulation of the cervix. For example, laparoscopic examination of the abdominal organs or of the female pelvic organs involves inserting a thin, lighted tube (called a laparoscope) through an incision in the abdominal wall to diagnose problems such as cysts, adhesions, fibroids, and infections. In the course of a laparoscopic examination, a clinician may wish to manipulate the cervix to observe different areas of the uterus and of the surrounding organs. Other gynecological procedures that may require a manipulation of the cervix include total or partial laparoscopic hysterectomy, tubal sterilization, removal of myomas or pelvic cysts, and a variety of radiographic procedures.

Manipulation of the cervix is typically accomplished by engaging the cervix with a manipulator device that is inserted into the external os of the cervix and passed through the cervical canal into the uterus. Some known cervical manipulators attempt to manipulate and seal the cervix by engaging the internal and external ora with opposing balloons. For example, U.S. Pat. No. 4,976,692 to Atad and U.S. Pat. No. 5,104,377 to Levine disclose devices that include a shaft having a first balloon at the distal end that is inserted into the uterus and a second balloon, that is disposed proximally of the first balloon, that is partially insertable into the cervical canal. The opposing pressures of the two balloons stabilize the cervix, but neither balloon is actually anchored to the cervix, Consequently, the two balloons must be manually adjusted in an attempt to properly seat the balloons against the internal and external ora, and application of excessive force on the proximal balloon may cause discomfort to the patient or an undesired withdrawal of the distal balloon even after inflation.

Other known devices attempt to manipulate the cervix by engaging the internal os with a balloon and the external os with a retainer. For example, U.S. Pat. No. 5,372,584 to Zink et al. discloses a device having a balloon at the distal end that is positioned against the internal os and a disk at the proximal end that is slidable against the external os, and U.S. Pat. No. 5,464,409 to Mohajer discloses a device that includes a balloon at the distal end that engages the internal os and a curved blade at the proximal end that engages the external os. The disk in Zink is not anchored to the cervix and the balloon must be manually adjusted in an attempt to properly seat the device against the internal and external ora, a process that is cumbersome and that may involve one or more resettings of the proximal stop to securely engage the cervix. Instead, the cervical blade in Mohajer requires skill to apply and may cause discomfort or even harm to the patient if improperly used.

Still other devices in the prior art attempt to manipulate the cervix by employing conical occluding members positioned against the cervical ora. For example, U.S. Pat. No. 5,540,658 to Evans et al. discloses a transcervical sealing device having a tubular body with an expandable conical member at the distal end and a rigid conical member at the proximal end that are positioned on the internal and external ora of the cervix and that are held in position by a spring-loaded mechanism. U.S. Pat. No. 5,935,098 to Blaisdell et al. discloses instead a device for manipulating the uterus that includes a balloon at the distal end of an inner catheter, that is positioned against the internal os, and an occluding member at the distal end of an outer catheter, that is positioned against the external os. Both Evans and Blaisdell require a maneuvering of the devices by the clinician to generate adequate pressures against the internal and external ora, either mechanically, as in Evans, or manually by maneuvering two catheters, as in Blaisdell. Additionally, the shape of the inner catheter in Blaisdell does not protect the uterine walls against accidental perforations that may be caused by the catheter tip and body, and the occluding member in Blaisdell can be retained against the external os only by continuous pressure applied by the clinician, because the smooth conical walls of the occluding member provide no gripping action against the external os.

Therefore, it also would be desirable to provide apparatus for manipulating the cervix that seat against the internal and external os without difficult manual adjustments and that minimize discomfort to the patient.

It also would be desirable to provide apparatus for manipulating the cervix that become anchored to the cervix without complex mechanical structures.

It further would be desirable to provide apparatus for manipulating the cervix that are simple to use and inexpensive to manufacture.

It still further would be desirable to provide apparatus for manipulating the cervix that minimize risk of perforations of the uterine walls during sounding or in the event of accidental contact.

### SUMMARY OF THE INVENTION

In view of the foregoing, it is an object of the present invention to provide apparatus for manipulating the cervix that may be operated easily and intuitively by a clinician.

It also is an object of the present invention to provide apparatus for manipulating the cervix that provide for a secure anchoring to the external and internal ora of the cervix.

It further is an object of the present invention to provide apparatus for manipulating the cervix that do not require complex mechanical structures.

It still further is an object of the present invention to provide apparatus for manipulating the cervix that can be manufactured at low cost and be discarded after use.

It yet further is an object of the present invention to provide apparatus for manipulating the cervix that are minimally invasive and that minimize discomfort to the patient.

It is another object of the present invention to provide apparatus for manipulating the cervix that minimize risk of perforations of the uterine walls during sounding or in the event of accidental contact.

It is yet another object of the present invention to provide apparatus for cervical stabilization that do not require a dilation of the cervix prior to use.

These and other objects of the present invention are accomplished by providing apparatus and methods for cervical manipulation that include an elongated member, a balloon that is disposed at the distal end of the elongated member and that is pressured against the internal os, and an applicator member that can be translated longitudinally and that can be anchored to the external os.

In one embodiment, the apparatus for uterine cervical manipulation includes an inner tubular member manufactured from a bendable, semi-rigid material that has an inflatable balloon disposed at the distal end and a flexible wire coupled to the wall of a lumen extending between the distal and proximal ends of the inner tubular member. The flexible wire may be attached to the wall of the lumen of the inner tubular member or may be embedded in the wall of the lumen. An atraumatic tip may be disposed at the distal end of the inner tubular member inside the inflatable balloon, or the inflatable balloon may be disposed proximally of the atraumatic tip.

The present embodiment also includes an outer tubular member, within which the inner tubular member reciprocates and which carries at the distal end an applicator member shaped to engage the external os. The applicator member is preferably frustoconical and carries one or more grooves or protrusions on its lateral surface that provide the applicator member with a screw-like pattern.

When a clinician desires to increase the rigidity of the inner tubular member or have at least a portion of the inner tubular member assume a predetermined angle, a rigid stylet can be inserted into the lumen of the inner tubular member. The rigid stylet may be manufactured from a metallic or plastic material.

In another embodiment, the apparatus for cervical manipulation includes a tubular member that is manufactured from a bendable, semi-rigid material and that has a flexible wire coupled to the wall of a lumen within the tubular member. An inflatable balloon is disposed at the distal end of the tubular member and an applicator member, shaped to engage the external os of a cervix, is disposed proximally of the inflatable balloon.

Methods of using the apparatus for cervical manipulation of the present invention are also provided.

### BRIEF DESCRIPTION OF THE DRAWINGS

The above and other objects and advantages of the invention will be apparent upon consideration of the following detailed description, taken in conjunction with the accompanying drawings, in which like reference numerals refer to like parts throughout, and in which:

FIGS. 1A-1B illustrate two variants of a first embodiment of the invention, the variant in FIG. 1A having an essentially spherical balloon and the variant in FIG. 1B having an essentially ellipsoidal balloon;

FIGS. 2A-2B illustrate two possible dispositions of a flexible wire in the embodiment of FIGS. 1A-1B;

FIGS. 3A-3B illustrate two possible dispositions of an atraumatic tip in the embodiment of FIGS. 1A-1B;

FIG. 4 illustrates a rigid stylet for use in combination with the embodiment of FIGS. 1A-1B;

FIGS. 5A-5B illustrate different disposition of a second lumen in the inner tubular member of the embodiment of FIGS. 1A-1B;

FIG. 6 illustrates the application of the embodiment of FIG. 1A in a patient;

FIG. 7 illustrates a locking member for use with the embodiments of FIGS. 1A-1B;

FIG. 8 illustrates a second embodiment of the invention; and

FIG. 9 illustrates a cross-section of the applicator member in the embodiment of FIG. 8.

### DETAILED DESCRIPTION OF THE INVENTION

The present invention is directed to apparatus and methods for manipulating the cervix that include an elongated member, a balloon disposed at the distal end of the elongated member that is pressured against the internal os, and an applicator member that can be translated longitudinally and be anchored to the external os.

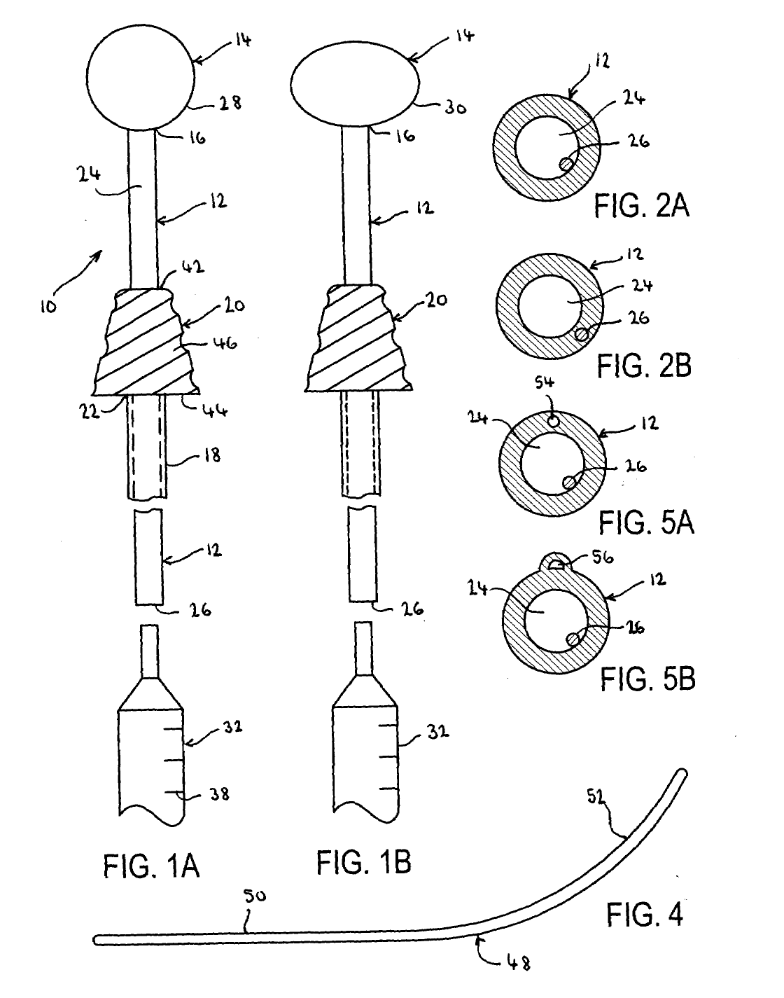

Referring first to FIGS. 1A-1B, a first embodiment of an apparatus for cervical manipulation constructed in accordance with the principles of the present invention is described. In its most basic components, apparatus for cervical manipulation 10 includes inner tubular member 12, having inflatable balloon 14 disposed at distal end 16, and outer tubular member 18, having applicator member 20 disposed at distal end 22.

Inner tubular member 12 is manufactured from a flexible or semi-rigid material, for example, from surgical-grade polypropylene. Lumen 24 extends longitudinally between distal end 16 and proximal end 26, and provides fluid communication between proximal end 26 and balloon 14. With further reference to FIGS. 2A-2B, flexible wire 26 is coupled to the wall of lumen 24, and is preferably attached to the wall of lumen 24, as shown in FIG. 2A, but in a variant of embodiment 10, flexible wire 26 may be embedded within the wall of lumen 24, as shown in FIG. 2B.

Within the context of the present specification, the term “flexible wire” is used to indicate a wire that bends under lateral pressure but that reacquires its original shape after lateral pressure is released. Such a flexible wire may be manufactured from a metallic material, for example, from steel, or from a non-metallic material, for example, from highly oriented polypropylene, or from a combination of metallic and non-metallic materials.

The presence of flexible wire 26 provides resilient properties to inner tubular member 26 when longitudinal pressure is applied on distal end 16. More specifically, flexible wire 26 causes inner tubular member 12 to bend without snapping under longitudinal pressure, which proves particularly advantageous during uterine sounding, as explained in greater detail below. Additionally, flexible wire 26 provides a mechanical reinforcement to inner tubular member 12, increasing longitudinal rigidity of inner tubular member 12 and allowing the manufacture of inner tubular member 12 with a thinner diameter than otherwise possible, thereby enabling the introduction of inner tubular member 12 into an undilated cervix.

Balloon 14 preferably has a volume after inflation of approximately 3 cc and may be manufactured from a variety of materials known in the art, for example, from medical-grade rubber, plastic, or from a composite material suitable for uterine applications. Balloon 14 may be shaped to acquire an essentially spherical shape 28 after inflation, as depicted in FIG. 1A, or an essentially ellipsoidal shape 30 having the major axis disposed perpendicular to the longitudinal axis of inner tubular member 12, as depicted in FIG. 1B. One skilled in the art will appreciate that balloon 14 may be shaped to acquire a variety of other shapes, which are within the scope of the present invention and which are not described here for the sake of brevity.

Balloon 14 is in flow communication with lumen 24 and with injection device 32 that is connected to distal end 26 and that supplies an inflation fluid to balloon 14, for example, a gas or a saline solution. Typically, inflation device 32 is a syringe that is connected to distal end 26 with devices or methods known in the art, for example, with a Luer-Lok fitting. Markings 38 may be disposed on injection device 32, to indicate the amount of fluid necessary to inflate balloon 14 by a predetermined diametral increment. Additionally, one or more apertutes (not shown) may be present on inner tubular member that are in fluid communication with an radio-opaque solution that may be dispensed inside the uterus.

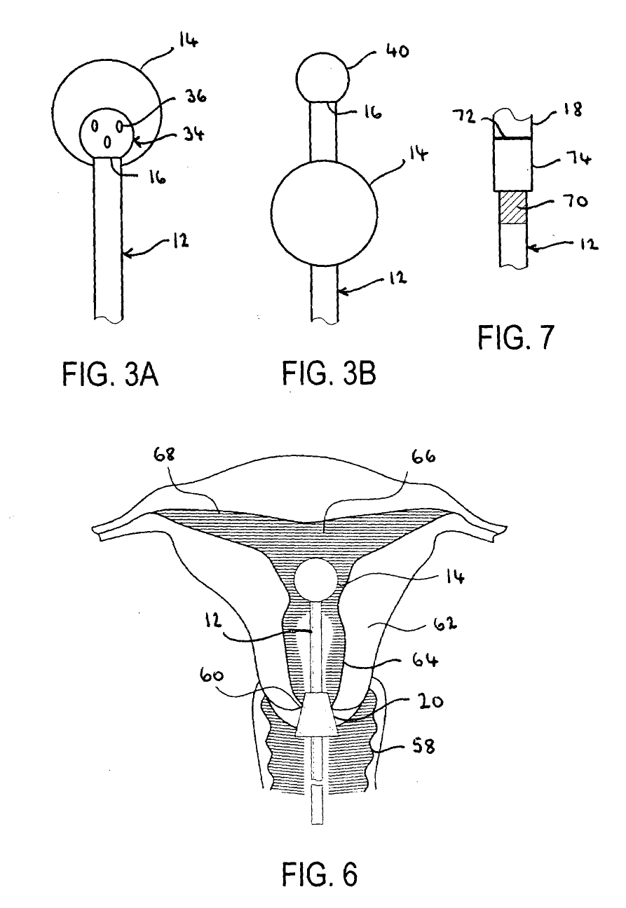

With further reference to FIGS. 3A-3B, atraumatic tip 34 may be disposed at distal end 16 and may be enclosed by balloon 14, as shown in FIG. 3A, in which case balloon 14 is inflated through one or more apertures 36 in atraumatic tip 34 that enable the passage of the inflating fluid. Alternatively, atraumatic tip 40 may be disposed at distal end 16 while balloon 14 is disposed in the distal portion of inner tubular member 12 proximally to atraumatic tip 40, as shown in FIG. 3B. In the latter configuration, balloon 14 is inflated through one or more apertures (not shown) on inner tubular member 12 that provide fluid communication with injection device 32. In another configuration (not shown), the atraumatic tip may be shaped as an integral extension of the balloon, and may or may not be inflatable.

During the sounding of the uterus (that is, during contact of apparatus 10 with the uterine walls to provide a clinician with information on shape, size and position of the uterus), atraumatic tip 34 or 40 protects the walls of the uterus against accidental perforation by virtue of its relatively enlarged shape and of flexible wire 26 disposed within lumen 24. In particular, upon contact of distal end 16 with a uterine wall, flexible wire 26 causes inner tubular member 26 to bend rather than retaining its relatively linear shape and eventually snapping under the applied pressure. At the same time, atraumatic tip 34 causes distal end 16 to slide away along the uterine wall rather than perforating the uterine wall.

Inner tubular member 12 reciprocates within outer tubular member 18, so that, during use by a clinician, applicator member 20 is disposed proximally of balloon 14. Applicator member 20 is preferably frustoconical in shape, with minor base 42 oriented in the direction of distal end 16 and with proximal base 44 oriented in the direction of proximal end 26. Applicator member 16 is dimensioned to fit within the external os of an undilated human cervix and is typically one centimeter long, with a diameter of seven millimeters at mid-shaft.

Applicator member 16 has a screw-like shape defined by one or more grooves 46 or protrusions carved on its lateral surface, which enable a secure anchoring of applicator member 20 to the cervix after insertion into the external os with a twisting motion. In order to facilitate insertion and reduce discomfort to the patient, the perimetral edge of minor base 42 is preferably rounded, as shown in FIGS. 1A-1B. Optionally, a control arm (not shown) may be attached to major base 44 to assist the clinician during the positioning of applicator member 20 in the external os.

The shape of applicator member 16 enables the clinician to use applicator member as a tenaculum that grasps the lower part of the cervix and moves it longitudinally or laterally as desired. Applicator member 20 is preferably manufactured from a resilient material, for example, from a silicone material, which provides for a less harsh contact with the external os than a rigid material such as a metallic or glass material, and which can be manufactured through a low cost industrial processes such as injection molding.

In certain situations, a clinician may find it desirable to operate with a stiffer inner tubular member 12, in order to achieve an easier insertion into the undilated cervix, and also may find it desirable to alter the curvature of inner tubular member 12, in order to achieve a desired angle of insertion. At the same time, a clinician may find it desirable to have inner tubular member 12 retain a flexible structure during part of the procedure, for example, during uterine sounding. To meet the above described requirements, inner tubular member 12 is manufactured from a flexible or semi-rigid material, as previously described, and may be stiffened by inserting a stylet through lumen 24.

FIG. 4 illustrates one type of stylet 48 that may be employed in conjunction with apparatus 10. Stylet 48 is manufactured from a rigid material, such as a metal or a rigid plastic, including polycarbonate. In one configuration, stylet 48 may be shaped to include a linear portion 50 having a typical length of 20 cm and an arched portion 52 having a typical length of 15 centimeters. The extremities of stylet 48 are preferably rounded, to facilitate insertion into lumen 22 and to avoid damage to bodily tissues in case of accidental contact.

When the use of stylet 48 is planned, alternative paths for supplying inflation fluid from injection device 32 to balloon 14 may be employed, to avoid loss of fluid from balloon 14 during the insertion of stylet 48. It should be noted, however, that such alternative paths may not be necessary, for example, when it is planned to use apparatus for cervical manipulation 10 first with stylet 48 disposed in lumen 24, for example during insertion through the cervix, and later to remove stylet 48 after insertion, for example for uterine sounding, leaving lumen 24 available for the passage of the inflating fluid.

When the above alternative paths are planned, separate lumen 54 may be embedded within the wall of inner tubular member 12, as shown in FIG. 5A, providing fluid communication between injection device 32 and balloon 14. In an alternative configuration, separate lumen 56 may be defined by a longitudinal cavity overlapping the wall of tubular member 12, as shown in FIG. 5B, providing fluid communication between injection device 32 and balloon 14.

Referring now to FIG. 6, a method of use of apparatus for cervical manipulation 10 is described. After vaginal canal 40 is spread using a standard vaginal speculum or a similar instrument (not shown), apparatus for cervical manipulation 10 is inserted through external os 60 of cervix 62. The thin diameter of inner tubular member 12 makes it possible to insert inner tubular member 12 through cervix 62 in undilated condition, and balloon 14 is maintained in a deflated condition as it travels through external os 60 and cervical canal 64, eventually entering uterine cavity 66. The clinician is then in a position to sound uterine cavity 66 by extending inner tubular member into uterine cavity 66 until contact with uterine fondus 68 is achieved. The presence of flexible wire 26 within lumen 24, and of atraumatic tip 34 or 40 at distal end 16, minimizes the risk of perforation of uterine fondus 68 in the event the clinician applies excessive pressure against uterine fondus 68, because inner tubular member 12 will bend and distal tip 16 will slide away along uterine fondus 68 rather than maintaining a concentrated load against uterine fondus 68.

In situations where atraumatic tip 34 or 40 is not present, or where the clinician prefers not to use it, balloon 14 may be inflated and operate as an atraumatic tip during sounding.

If the clinician wishes to operate with an inner tube member 12 having greater rigidity, and/or to shape inner tubular member 12 to a contour that may provide easier maneuverability, stylet 48 can be disposed within inner tubular member 12 and be retracted before sounding of the uterine cavity 66.

As a next step, outer tubular member 18 is advanced in the direction of external os 60 and inserted into external os 60 by twisting applicator member 20 with a screw-like motion, achieving a secure anchoring of applicator member 12 to external os 60.

Balloon 14 is successively inflated, typically to a diameter of approximately 10 mm, by injecting a fluid into lumen 24 through proximal end 26. For example, a saline solution may be injected into lumen 24 from a syringe 32 connected to distal end 26, and the appropriate amount of fluid that needs to be injected to have balloon 14 progressively inflate to a certain diameter may be determined by using markings 38 on syringe 32 as a guide. Alternatively, the saline solution may reach balloon 14 through lumen 54 or lumen 56 when the embodiments shown in FIG. 5A or FIG. 5B are utilized.

The inflation of balloon 14 and the inward pressure applied by the clinician causes balloon 14 to come in close contact with the internal os of cervix 62. As a consequence, cervix 62 becomes stabilized by being held by the combined, opposing pressures applied by applicator member 20 against external os 60 and of balloon 14 against the internal os.

After applicator member 20 and balloon 14 are positioned in the desired locations, inner tubular member 12 is locked against outer tubular member 18 to maintain the grip on cervix 64. One of the many possible locking systems and methods known in the art may be employed, for example, the clinician may maintain sufficient hand pressure against both inner tubular member 12 and outer tubular member 18 or, alternatively, a locking member may be employed.

One such locking member 74 is illustrated in FIG. 7, which shows inner tubular member 12 comprising threaded portion 70, situated in the area where proximal end 72 of outer tubular member 18 is expected to be positioned when the desired grip on cervix 62 is achieved. Locking member 74, having a threaded inner lumen, is then slid over inner tubular member 12 and screwed over threaded portion 70, in the same fashion as a nut over a bolt, to abut proximal end 72 and maintain it in the desired location. One skilled in the art will recognize that a plurality of alternative locking systems may be envisioned that fall within the scope and spirit of the present invention, including but not limited to protrusions that snap out of inner tubular member 12 or locking pins inserted through inner tubular member 12.

When cervix 48 needs no longer to be manipulated, balloon 14 is deflated and applicator member 16 is de-anchored from external os 42, enabling the clinician to slide apparatus 10 out the genital area of the patient.

One skilled in the art will recognize that the above described method steps may be performed in different orders, which are still within the scope and spirit of the present invention. To mention just one example, balloon 14 may be inflated and pressed against the internal os before (rather than after) anchoring applicator member 20 into external os 60.

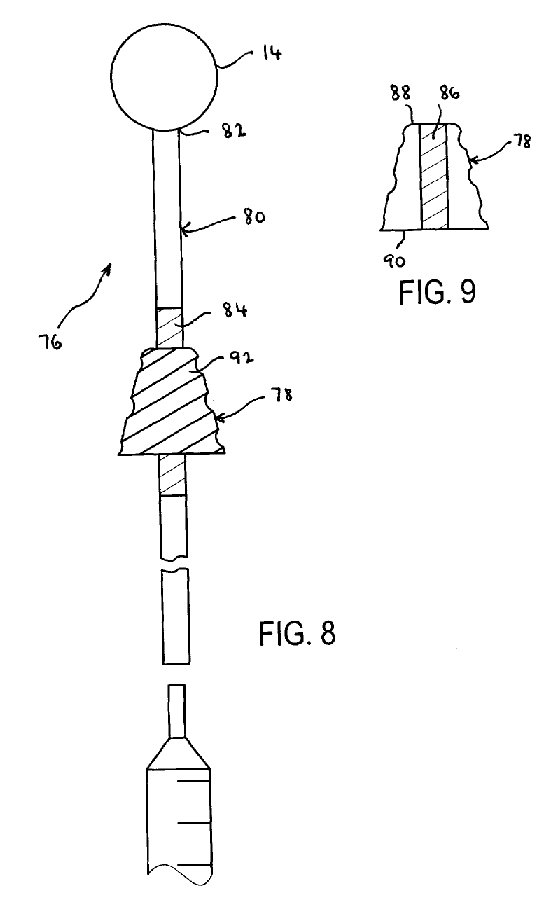

Referring now to FIG. 8, a second embodiment of an apparatus for cervical manipulation constructed in accordance with the principles of the present invention is described. Apparatus 76 is structured in a manner similar to apparatus 10, and the same components will be identified hereinafter by the same reference numerals. The main difference between apparatus 10 and apparatus 76 consists in having balloon 14 and applicator member 78 both disposed on apparatus 76 on a single tubular member 80. More specifically, balloon 14 is disposed at distal end 82 of tubular member 80 and applicator member 78 is disposed proximally of balloon 14, along threaded portion 84.

Applicator member 78 includes an inner lumen 86 that extends longitudinally between minor base 88 and major base 90 and that carries a threaded pattern that matches the pattern of threaded portion 84. After tubular member 80 has been inserted through cervical canal 64 and into uterine cavity 66, balloon 14 is inflated and applicator member 78 is advanced towards external os 60, first by sliding applicator member 78 along the proximal portion of tubular member 80, and then, upon reaching the proximal end of threaded portion 84, by twisting applicator member 78 along threaded portion 84 until grooves 92 on the lateral surface of applicator member 78 become anchored in external os 60.

Therefore, cervix 62 become stabilized by the opposing pressures of balloon 14 and of applicator member 78, and applicator member 78 remains locked in the desired position along tubular member 80 by the mating of the threads on threaded portion 84 on tubular member 80 with the threads on lumen 86. Preferably, the pitch and angle of threaded portion 84 and lumen 86 are the same as that of grooves 92.

While embodiments of the invention have been described above, it will be apparent to one skilled in the art that various changes and modifications may be made. The appended claims are intended to cover all such changes and modifications that fall within the true spirit and scope of the invention.

What is claimed is:

1. An apparatus for cervical manipulation comprising:
an inner tubular member having a distal end, a proximal end, and a first lumen therebetween, the inner tubular member being manufactured from a bendable, semi-rigid material, a flexible wire being coupled to the wall of the first lumen;
an inflatable balloon disposed at the distal end of the inner tubular member, the inflatable balloon being in fluid communication with an injection device;
an outer tubular member having a distal end, a proximal end, and a second lumen therebetween, the inner tubular member reciprocating in the second lumen; and
an applicator member disposed at the distal end of the outer the tubular member, the applicator member being situated proximally of the inflatable balloon, the applicator member being shaped to engage the external os of a cervix.

2. The apparatus of claim 1, wherein the flexible wire is attached to the wall of the first lumen.

3. The apparatus of claim 1, wherein the flexible wire is embedded in the wall of the first lumen.

4. The apparatus of claim 1, wherein the balloon is essentially spherical in shape.

5. The apparatus of claim 1, wherein the balloon is essentially ellipsoidal in shape.

6. The apparatus of claim 1, wherein the applicator member is frustoconical in shape and carries one or more spirally disposed grooves or protrusions on its lateral surface.

7. The apparatus of claim 1, wherein the applicator member is manufactured from a resilient material.

8. The apparatus of claim 1, wherein fluid communication is provided through the first lumen.

9. The apparatus of claim 1, wherein fluid communication is provided through a third lumen disposed in the wall or along the lateral surface of the inner tubular member.

10. The apparatus of claim 1, further comprising an atraumatic tip disposed at the distal end of the inner tubular member, the atraumatic tip being disposed within the inflatable balloon.

11. The apparatus of claim 1, further comprising an atraumatic tip disposed at the distal end of the inner tubular member, the inflatable balloon being disposed proximally of the atraumatic tip.

12. The apparatus of claim 1, further comprising a rigid stylet insertable into the first lumen.

13. The apparatus of claim 12, wherein the stylet provides the tubular member with a predetermined curvature.

14. The apparatus of claim 1, wherein the outer tubular member is lockable against the inner tubular member.

15. The apparatus of claim 14, wherein the outer tubular member is lockable by disposing a locking member adjacent to the proximal end of the outer tubular member.

16. The apparatus of claim 15, wherein the locking member includes a threaded lumen, and wherein the threaded lumen engages a threaded portion disposed on the inner tubular member.

17. A method of manipulating a uterine cervix, the method comprising:
providing an inner tubular member manufactured from a bendable, semi-rigid material and having a first lumen, an inflatable balloon being disposed at the distal end of the inner tubular member, a flexible wire being coupled to the wall of the first lumen, the inflatable balloon being in fluid communication with an injection device;
providing an outer tubular member having a second lumen and an applicator member disposed at the distal end of the outer the tubular member, the applicator member being shaped to engage the external os of the cervix, the inner tubular member reciprocating in the second lumen; and
inserting the distal end of the inner tubular member inside the uterus;
anchoring the applicator member to the external os;
inflating the balloon and contacting the balloon against the internal os of the cervix; and
locking the outer tubular member against the inner tubular member.

18. The method of claim 17, wherein the applicator member has a frustoconical shape and one or more grooves or protrusions spirally disposed on its external surface.

19. The method of claim 17, wherein inserting the inner tubular member into the cervix comprises inserting a rigid stylet within the first lumen.

20. The method of claim 17, wherein locking the outer tubular member with the inner tubular member comprises disposing a locking member adjacent to the proximal end of the outer tubular member.

21. The method of claim 17, further comprising the step of sounding the uterus by extending the distal end of the inner tubular member inside the uterus until contact is made with a uterine wall.

22. An apparatus for cervical manipulation comprising:
a tubular member having a distal end, a proximal end, and a lumen therebetween, the tubular member being manufactured from a bendable, semi-rigid material, a flexible wire being coupled to the wall of the lumen;
an inflatable balloon disposed at the distal end and in fluid communication with an injection device; and
an applicator member disposed on the tubular member proximally of the inflatable balloon, the applicator member being shaped to engage the external os of a cervix.

23. The apparatus of claim 22, wherein the balloon is essentially spherical in shape.

24. The apparatus of claim 22, wherein the balloon is essentially ellipsoidal in shape.

25. The apparatus of claim 22, wherein the flexible wire is attached to the wall of the lumen.

26. The apparatus of claim 22, wherein the flexible wire is embedded in the wall of the lumen.

27. The apparatus of claim 22, wherein the applicator member is frustoconical in shape and carries one or more spirally disposed grooves or protrusions on its lateral surface.

28. The apparatus of claim 22, wherein the applicator member has a threaded lumen, and wherein the threaded lumen engages a threaded portion on the inner tubular member.

29. The apparatus of claim 22, wherein the applicator member is manufactured from a resilient material.

30. The apparatus of claim 22, wherein fluid communication is provided through the lumen.

31. The apparatus of claim 22, wherein fluid communication is provided through a second lumen disposed in the wall or along the lateral surface of the inner tubular member.

32. The apparatus of claim 22, further comprising an atraumatic tip disposed at the distal end of the tubular member, the atraumatic tip being disposed within the inflatable balloon.

33. The apparatus of claim 22, further comprising an atraumatic tip disposed at the distal end of the tubular member, the inflatable balloon being disposed proximally of the atraumatic tip.

34. The apparatus of claim 33, wherein the atraumatic tip and the inflatable balloon are manufactured as a single component.

35. The apparatus of claim 22, further comprising a rigid stylet insertable into the lumen.

36. The apparatus of claim 35, wherein the rigid stylet provides the tubular member with a predetermined curvature.
